(19) United States
(12) Patent Application Publication
(10) Pub. No.: US 2008/0193500 A1
(43) Pub. Date: Aug. 14, 2008

(54) Effect of LMP-1 overexpression on large and small proteoglycans of the disc

(76) Inventors: William F. McKay, Memphis, TN (US); Jeffrey C. Marx, Germantown, TN (US); Susan J. Drapeau, Cordova, TN (US)

Correspondence Address: Medtronic;Spinal and Biologics, 2600 Sofamor Danek Drive, Memphis, TN, 38132, US

(21) Appl. No.: 11/705,942

(22) Filed: Feb. 14, 2007

(51) Int. Cl. A61K 31/7088; A61K 38/16; A61K 9/00; A61P 19/00

(52) U.S. Cl. 424/423; 424/93.7; 514/12; 514/44

(57) ABSTRACT

A method of treating a disease of an intervertebral disc characterized by an altered amount of at least one proteoglycan, collagen, or heteropolysaccharide in an extracellular matrix of the intervertebral disc are provided. The methods comprise administering to cells of the intervertebral disc having the disease a composition comprising a nucleic acid sequence encoding an amino acid sequence at least 70% identical to an LMP protein or fragment thereof.

### FIELD OF INVENTION

This invention is related to the field of correcting the amounts of proteoglycans, collagen, and/or heteropolysaccharides which are altered in course of diseases of the intervertebral disc.

### BACKGROUND

The progressive degeneration of intervertebral discs with age is believed to be associated with a decrease in cell density and a alteration in synthesis of cartilage-specific matrix components, especially proteoglycans. See, e.g., S. J. Lipson & H. Muir H., Spine 6: 194-210 (1981); A. G. Nerlich et al., Spine 22: 2781-95 (1997); R. H. Pearce et al., J. Orthop. Res. 5: 198-205 (1987). This may cause the disc to be more susceptible to bio-mechanical injury and degeneration.

Research into growth factors to stimulate matrix components has focused on aggrecan which, along with versican, are large porteoglycans found in the disc. However, relatively little data is available on the other proteoglycans, such as biglycan, decorin, lumican, and fibromodulin. These small proteoglycans bind to collagens, growth factors, and other matrix components and are believed to play important roles in regulation and post-injury repair of the extracellular matrix of the intervertebral discs. As a disc degenerates the levels of type II collagen decrease. The collagen provides a 3-dimensional matrix for cell attachment and proliferation. The proteoglycans produced by the cells increase water uptake and nucleus volume increase required to maintain proper disc height.

Previous research has shown that Lim Mineralization Protein (LMP) can increase aggrecan and collagen synthesis. However, the effect of LMP on other proteoglycans and other components of the extracellular matrix has not been established.

Degenerated discs are a significant source of spine-related pain. Amongst sufferers of chronic pain, spine-related problems constitute the bulk of such complaints. Spinal pain has been estimated to exist in as much as 66% of the general population. Beyond the substantial discomfort that back pain inflicts upon individuals, spine-related pain also incurs heavy societal costs. For example, as many as one million spine surgeries, and as many as five million interventional procedures, are estimated to be performed in the United States each year. Well beyond the purely medical and psychological burdens imposed by such procedures, the subsequent social costs related to productivity, disability compensation and lost taxes are substantial.

Accordingly, it would be desirable to have a method of modulating proteoglycan, collagen and/or heteropolysaccharides synthesis by the appropriate cells, such as, for example, cells of the nucleus pulposus, cells of the annulus fibrosus, and cells of the intervertebral disc.

### SUMMARY OF INVENTION

The instant invention addresses these and other needs by providing novel methods of treating disc diseases characterized by altered amounts of heteropolysaccharides proteoglycans, and collagens.

In one aspect, the invention provides a method of treating a disease of an intervertebral disc characterized by an altered amount of at least one proteoglycan or at least one collagen or at least one heteropolysaccharide in an extracellular matrix of the intervertebral disc, comprising a step of increasing an amount of a composition comprising an amino acid sequence at least 70% identical to an LMP protein or a fragment thereof in a cell, wherein the amino acid sequence is capable of regulating the amount of the at least one proteoglycan or the at least one collagen or the at least one heteropolysaccharide produced by said cell. In different embodiments of the invention, the concentration of the at least one proteoglycan is decreased in the diseased disc, and the at least one proteoglycan is selected from the group consisting of aggrecan, versican, lumican, and a combination thereof. In another embodiment, the amount of at least one collagen is decreased in the diseased disc and the composition is capable of increasing the amount of the at least one collagen. In different embodiments, suitable examples of the at least one collagen are a type I collagen protein, a type II collagen protein, a type III collagen protein, and a type X collagen. In a specific embodiment, the at least one collagen is the type I collagen protein the type II collagen protein the type X collagen protein, or a combination thereof. In another embodiment, the concentration of the at least one proteoglycan is increased in the diseased disc, and the at least one proteoglycan fibromodulin. In yet another embodiment of the invention, the heteropolysaccharide is a glycosaminoglycan, or, more specifically, a sulfated-glycosaminoglycan.

### DETAILED DESCRIPTION OF PREFERRED EMBODIMENTS

For the purposes of this invention, the following non-limiting definitions are provided:

The terms “allograft” and “allogeneic” refer to a graft of tissue obtained from a donor of the same species as, but with a different genetic make-up from, the recipient, as a tissue transplant between two humans. The terms “autograft” and “autogeneic” refer to being derived or transferred from the same individual's body.

The terms “xenograft” and “xenogeneic” refer to being derived from a donor of a different species than recipient.

The terms “intervertebral disc” and “intervertebral disc tissue” include the endplate, the nucleus pulposis and/or the annulus fibrosis.

The term “vector” refers to a nucleic acid assembly capable of transferring gene sequences to target cells (e.g., viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a nucleic acid sequence of interest and which can transfer gene sequences to target cells. Thus, the term includes cloning and expression vehicles, as well as viral vectors.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “practitioner” refers to a person who uses methods and compositions of the current invention on the patient. The term includes, without limitations, doctors, nurses, scientists, and other medical or scientific personnel.

The term “multipotent cells” refers to cells capable of differentiation into more than one cell type. As used herein, multipotential cells include but are not limited to mesenchymal cells.

The term “microspheres” refers to generally spherical particles 10 μm-100 μm in size. Microspheres may comprise, for example, a hollow space encapsulated by lipids, polymers, at least one surfactant, or any combination thereof, wherein the hollow space comprises therapeutic agent, such as at least one additive. In different embodiments, microspheres may include microbubbles and liposomes.

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (5th Ed. 2002).

Cells

Research into LMP as a possible agent for proteoglycan and BMP upregulation has previously only focused on intervertebral disc cells or bone marrow cells. However, this invention is not limited to these cell types only. Multiple cell types are useful for this invention, such as for example, different types of multipotential cells. The multipotential cells can be derived from various tissue sources in the body. In different embodiments the cell population may be isolated from a living donor or a cadaver tissue source. Such tissue sources include, but are not limited to, adipose tissue, muscle tissue, peripheral blood, cord blood, blood vessels, skeletal muscle, skin, liver, and heart. In the practice of the invention, the cell source may include whole cells, concentrated cells, filtered cells, separated cells, and cell populations isolated and culture-expanded from a tissue source.

In one embodiment, the cells are bone marrow cells. These cells are readily available from an accessible source and can be harvested from human donors with minimal morbidity. If the bone marrow cells are used in the practice of the invention, the cell source may be whole bone marrow, concentrated bone marrow, filtered bone marrow, separated bone marrow cells, and cell populations isolated and culture-expanded from the bone marrow source. Importantly, using bone marrow cells obviates the practical problems of autologous or allogeneic disc cell harvest and greatly shortens the time required for cell preparation in the clinical transplantation procedure.

The cells of the present invention may be derived not only from an autogeneic source, but also from allogeneic or even xenogeneic sources. A person of ordinary skill in the art will understand, however, that using autogeneic source of the cells will minimize chance of immune response and other unwelcome side effects to the composition of the present invention.

Further, a person of ordinary skill in the art will understand that even not fully differentiated cells may be administered to the subject in need thereof. Certain physical and chemical characteristics of the placement area within the subject's body will cause the cells which are not not fully differentiated to differentiate into intervertebral disc cells and thereby repair or form intervertebral disc tissue in the subject. Among those physical and chemical characteristics are compressive forces, shear forces, low oxygen tension (between about 1% and about 5%), relatively high pressure. A suitable non-limiting example of a combination of such conditions includes 1,800 cycles/day or 7,200 cycles/day of 1 Hz sinusoidal hydrostatic compression to 5 MPa. Elder et al., Biomech Model Mechanobiol. 3(3):141-6 (2005). Epub Jan. 25, 2005. Intervertebral disks and joints have such characteristics, and therefore will stimulate the cells to differentiate into intervertebral disc cells if the cells are not fully differentiated at the time of the administration to the subject.

Compositions and Intracellular Delivery Thereof.

As discussed above, in one aspect, the instant invention provides a method of treating a disease of an intervertebral disc characterized by an altered amount of at least one proteoglycan or at least one heteropolysaccharide in an extracellular matrix of the intervertebral disc, comprising a step of increasing an amount of a composition comprising an amino acid sequence at least 70% identical to an amino acid sequence encoding a LMP protein or a fragment thereof in a cell, wherein the amino acid sequence is capable of regulating the amount of the at least one proteoglycan or the at least one collagen or the at least one heteropolysaccharide, produced by said cell. A person of the ordinary skill in the art will appreciate that in different embodiments of the invention, the amino acid sequence may be at least 75% identical, or at least 80% identical, or at least 85% identical, or at least 90% identical or at least 95% identical or 100% identical to the amino acid sequence encoding the LMP protein or the fragment thereof.

Different members of the LMP family are suitable for the instant invention. Currently, LPM-1, LMP-2, LMP-3, and LMP-1s proteins have been identified. All these proteins are suitable for the methods of the instant invention and included within the meaning of the term “LMP protein”. The amino acid sequences for the LMP-1, LMP-2, LMP-3, and LMP-1s (SEQ. ID. NO.: 1, SEQ. ID. NO.: 2, SEQ. ID. NO.: 3, and SEQ. ID. NO.: 4, respectively) are known in the art.

A person of the ordinary skill in the art will undoubtedly appreciate that multiple methods exist for increasing the amount of the amino acid sequence which is at least 70% identical to the amino acid sequence of the LMP protein or the fragment thereof in the cell.

In one embodiment, the cell is contacted with a composition comprising the amino acid sequence which is at least 70% identical to the amino acid sequence encoding the LMP protein or the fragment thereof. Upon contact, the amino acid sequence which is at least 70% identical to the amino acid sequence encoding the LMP protein or the fragment thereof will enter the cell, thus increasing its amount within the cell. The composition may further comprise a suitable carrier or diluent. Further, the composition may comprise transportation means. As a suitable non-limiting example, the transportation means may comprise a TAT domain (SEQ. ID. NO.: 5, RKKRRQRRR) or a PTD domain (including, without limitations RQIKIWFQNRRMKWKK, GRKKRRQRRRPPQC, GWTLNSAGYLLKINLKALAALAKKIL, RRRRRRRRR, PIRRRKKLRRLK, RRQRRTSKLMKR, SRRKRQRSNMRI, SFHQFARATLAS, DPATNPGPHFPR, and TLPSPLALLTVH, referred to as SEQ. ID. NOs: 6-15, respectively), which can be derived, for example, from a viral protein. The TAT or the PTD domain will be fused with the amino acid sequence which is at least 70% identical to the amino acid sequence encoding the LMP protein or the fragment thereof. A person of the ordinary skill in the art will have the expertise to successfully fuse the TAT or the PTD domain with the desired amino acid sequence without disturbing the required activity of the LMP protein or the fragment thereof. The nucleic acid sequences for different LMP proteins, such as the LMP-1 protein (e.g., SEQ. ID. NO. 16), the LMP-2 protein (e.g., SEQ. ID. NO. 17), the LMP-3 protein (e.g., SEQ. ID. NO. 18), and the LMP-1s (e.g., SEQ. ID. NO.: 19), are known in the art and available from different sources including, without limitation, Genbank and different published non-patent and patent references. See, e.g., U.S. Pat. No. 6,300,127, incorporated herein to the extent it is not inconsistent with the instant disclosure. Further, the composition may comprise the desired amino acid sequence encapsulated in a compound or a mixture of compounds which promotes delivery of proteins into a cell. For example, the desired amino acid sequence may be encapsulated into liposomes or microbubbles or enveloped in any other lipophilic or ampiphilic polymer or a surfactant. A combination of the lipophilic or ampiphilic polymer or the surfactant and the TAT or the PTD domain is also possible.

The desired amino acid sequence, optionally, in combination with the TAT or the PTD domain, may be obtained by multiple methods. It can be synthesized in vitro, or produced by an expression system, including, without limitation, eukaryotic and prokaryotic expression systems, such as bacterial, yeast, insect, or mammalian expression systems. If glycosylation of the desired nucleic system is desired or required, a eukaryotic system is preferred. If glycosylation is unnecessary and/or undesired, the prokaryotic expression system may be used.

In another embodiment, the composition may comprise the nucleic acid sequence encoding the amino acid sequence which is at least 70% identical to the amino acid sequence encoding the LMP protein or the fragment thereof. In such embodiment, the nucleic acid sequence enters the cell and then the amino acid sequence which is at least 70% identical to the amino acid sequence encoding the LMP protein or the fragment thereof is expressed in the cell. A person of the ordinary skill in the art will recognize that the nucleic acid sequence may be included within a vector, such as, for example, a plasmid vector, or a viral vector.

In one set of embodiments, the vector is a viral vector, including, without limitations, alphaviral, lentiviral, retroviral, adenoviral, adeno-associated viral (AAV) (Williams and Koch, Annu. Rev. Physiol. 66:49 (2004); del Monte and Hajjar, J. Physiol. 546.1:49 (2003). Both adenoviral and AAV vectors have been shown to be effective at delivering transgenes (including the nucleic acid sequence encoding the amino acid sequence which is at least 70% identical to the amino acid sequence for the LMP protein or the fragment thereof) into mammalian cells, (e.g., Iwanaga et al., J. Clin. Invest. 113:727 (2004); Seth et al., Proc. Natl. Acad. Sci. USA 101:16683 (2004); Champion et al., Circulation 108:2790 (2003); Li et al., Gene Ther. 10:1807 (2003); Vassalli et al., Int. J. Cardiol. 90:229 (2003); del Monte et al., Circulation 105:904 (2002); Hoshijima et al., Nat. Med. 8:864 (2002); Eizema et al., Circulation 101:2193 (2000); Miyamoto et al., Proc. Natl. Acad. Sci. USA 97:793 (2000); He et al., Circulation 100:974 (1999). Recent reports have demonstrated the use of AAV vectors for sustained gene expression in mouse and hamster cells for over one year (Li et al., Gene Ther. 10:1807 (2003); Vassalli et al., Int. J. Cardiol. 90:229 (2003)). In particular, expression vectors based on AAV serotype 6 have been shown to efficiently transduce muscle cells (e.g., Blankinship et al., Mol. Ther. 10:671 (2004)). The present invention also provides for the use of coxsackie viral vectors for delivery of the nucleic acid sequence encoding the amino acid sequence which is at least 70% identical to the amino acid sequence for the LMP protein or the fragment thereof.

Further, the person of the ordinary skill in the art will appreciate that the nucleic acid sequence may be placed under control of a promoter. The promoter may be tissue specific or constitutively active, such as, for example, a CMV promoter or a TK promoter, or an RSV promoter.

Yet further, the composition comprising the desired nucleic acid sequence may further comprise a transportation means. In one embodiment, the transportation means comprises liposomes or other compositions which increase the penetration of the desired nucleic acid into the cell of choice, as described above. In another embodiment, the nucleic acid sequence may be fused to a nucleic acid sequence encoding a functional TAT or PTD domain. The construction of the desired nucleic acid sequence (including optional nucleic acid sequences for TAT and/or PTD domains, promoters, and subcloning of the resulting nucleic acid sequence into a vector) is within the expertise of the person of the ordinary skill in the art and may be performed usual methods and techniques employed by molecular biologists and described, for example, in Sambrook et al., Molecular Cloning, A Laboratory Manual (3d ed. 2001) and Ausubel et al., Current Protocols in Molecular Biology (1994).

Suitable methods of introducing exogenous nucleic acid sequences are described in Sambrook and Russel, Molecular Cloning: A Laboratory Manual (3rd Ed., 2001), Cold Spring Harbor Press, NY. These methods include, without limitation, physical transfer techniques, such as, for example, microinjection or electroporation; transfections, such as, for example, calcium phosphate transfections; membrane fusion transfer, using, for example, liposomes; and viral transfer, such as, for example, the transfer using DNA or retroviral vectors. Other methods for introducing the nucleic acid sequences of the present invention into suitable cells, such as, for example, electroporation (see, e.g., Iversen et al., Genetic Vaccines and Ther. 3: 2-14 (2005)) will be apparent to a person of ordinary skill in the art. All such methods are within the scope of the present invention. The person of the ordinary skill in the art will select the suitable method of delivering the composition of the present invention depending on whether the cell is located in vivo or in vitro.

Additives

In different embodiments of the invention, the composition may further comprise at least one additive. Suitable non-limiting examples of additives include, without limitation, analgesics, anesthetics, antibiotics, anti-inflammatory compounds, radiocontrast media, and the like.

Anti-inflammatory compounds include both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Rainsford, Anti-inflammatory and Anti-Rheumatic Drugs, Vol. I-III, CRC Press, Boca Raton, (1985), and Scherrer, et al., Anti-inflammatory Agents, Chemistry and Pharmacology 1, Academic Press, New York (1974), each incorporated herein by reference.

Mixtures of these non-steroidal anti-inflammatory compounds may also be employed, as well as the pharmacologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms). Suitable non-limiting examples of such compounds include candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable analgesics include, without limitation, non-steroid anti-inflammatory drugs, non-limiting examples of which have been recited above. Further, analgesics also include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003)Pharmacological Reviews, 55:1-20, the content of which is incorporated herein by reference.

The at least one additive may also include a radiocontrast agent to verify the placement and/or the distribution of the composition in the target area. Suitable radiocontrast agents include barium and iodine compounds, metal ions, nitroxides, and gadolinium complexes, such as gadodiamine.

Sustained Release

In yet another embodiment, the composition, whether comprising the desired amino acid sequence or the desired nucleic acid sequence and, optionally, at least one additive, is in a sustained-release formulation. Such embodiment is especially preferable when the composition is delivered to the cell in vivo, e.g., within the body of the patient. Sustained-release formulations, include, for example, microspheres comprising a biodegradable polymer. Suitable examples of the biodegradable polymers suitable for the present invention include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

Delivery of the Composition to the Target Area

The composition of the present invention may be delivered to the cell by a plurality of methods. For example, in one embodiment, the composition may be delivered via a catheter placed adjacent to the cell. The catheter may be semi-permanently positioned in the desired area, such that the practitioner will not need to remove the catheter between the administrations of the composition to the cells in the course of treatment. Yet, after the treatment has been accomplished, the catheter may be withdrawn from the patient's body. The catheter may optionally be connected to a reservoir containing the composition and further comprising a timer or any other mechanism to control the dose of the composition released into the target area. In another embodiment, the composition may be delivered by an injection from a syringe.

The composition may also be delivered into the desired part of the subject's body (e.g., joint or intervertebral disk), by intramuscular, intravenous, intramedullary, or intraarticular injection, or any combination thereof.

In another embodiment, the composition may be incorporated within an intervertebral disc implant or a nucleus pulposus implant. In one embodiment, the composition may be incorporated simply by soaking the implant in the solution comprising the composition of the instant invention. In another embodiment, the composition may be dripped, injected, sprayed, or brushed onto the implant. When placed into the target area (e.g., within the patient's body), the implant will release the composition. This set of embodiments is especially advantageous when the cell is located in vivo.

In another set of embodiments, composition of the instant invention is introduced to the cells in vitro, e.g., in culture. The cells may then be cultured for additional amount of time. Optionally, at this state, the cells may be sorted, for example, by using a cell sorter, to increase the relative ratio of the cells expressing the LMP protein or the fragment thereof.

After the practitioner has sufficient amount of cells, he can resuspend the cells and introduce the media comprising the cells to the implant. In one embodiment, the media comprising cells may be dripped onto the implant while optionally being gently agitated to promote homogenous distribution of the cells throughout the volume of the medium. A person of the ordinary skill in the art will appreciate that other methods exist to incorporate the cells of the instant invention into a suitable implant (e.g., the nucleus pulposus implant, or the intervertebral disc implant).

In embodiments disclosed above, the implant may be formed of a solid material, including, without limitation, polymethylmethacrylate, silicones, polyurethanes, polyvinyl alcohol, polyamides, aromatic polyamide, polyethers, polyester liquid crystal polymers, ionomers, poly(ethylene-co-methacrylic) acids, polybutylene terephtalate (PBT), polycarbonates, polyaminocarbonates, lactic acid, glycolic acid, lactide-co-glycolides, anhydrides, orthoesters, caprolactone, epoxy, and any combinations thereof. Implants formed of these and other suitable solid materials may be used both to deliver the composition to the cell located in vivo and to deliver the cell with the increased amount of the desired amino acid sequence to the target area.

In other set of embodiments, the implant comprises a liquid composition which solidifies in vivo e.g., upon injection into the patient's body. Suitable materials which solidify in vivo include, without limitation, polysaccharides, proteins, polyphosphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), sulphonated polymers, poly(N-vinyl-2-pyrrolidone), polyethylene glycol, polyethyleneoxide, poly(2-hydroxy ethyl methacrylate), copolymers of acrylates with N-vinyl pyrrolidone, N-vinyl lactams, and any combination thereof. Implants which solidify in vivo are suitable for the embodiments, wherein the composition is delivered to the cell located in vivo.

In another embodiment, the cells may be injected into the target area by any of the methods described above (e.g., an injection by a syringe or delivery via a catheter).

The patent and scientific literature referred to herein establishes the knowledge that is available to those with skill in the art. All U.S. patents and published or unpublished U.S. patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the claims.

1. A method of treating a disease of an intervertebral disc characterized by an altered amount of at least one proteoglycan, at least one collagen, or at least one heteropolysaccharide in an extracellular matrix of the intervertebral disc, comprising a step of increasing an amount of a composition comprising an amino acid sequence at least 70% identical to an LMP protein or a fragment thereof in a cell, wherein the amino acid sequence is capable of regulating the amount of the at least one proteoglycan, the at least one collagen, or the at least one heteropolysaccharide produced by said cell.

2. The method of claim 1, wherein the step of increasing the amount of the amino acid sequence at least 70% identical to the LMP protein or a fragment thereof comprises administering a nucleic acid sequence encoding the amino acid sequence at least 70% identical to the LMP protein or the fragment thereof.

3. The method of claim 2, wherein the nucleic acid sequence is included within a vector.

4. The method of claim 3, wherein the vector is a viral vector.

5. The method of claim 4, wherein the viral vector is an adenoviral vector.

6. The method of claim 1, wherein the increasing the amount of the amino acid sequence at least 70% identical to the LMP protein or the fragment thereof increases the amount of the at least one proteoglycan or the at least one collagen present in a decreased amount in the intervertebral disc having the disease.

7. The method of claim 6, wherein the at least one proteoglycan is selected from the group consisting of aggrecan, versican, lumican, and a combination thereof.

8. The method of claim 6, wherein the at least one collagen is a type I collagen protein a type II collagen protein a type X collagen protein or a combination thereof.

9. The method of claim 1, wherein the increasing the amount of the amino acid sequence at least 70% identical to the LMP protein or the fragment thereof decreases the amount of the at least one proteoglycan present in an increased amount in the intervertebral disc having the disease.

10. The method of claim 9, wherein the at least one proteoglycan is fibromodulin.

11. The method of claim 1, wherein the nucleic acid sequence is in a sustained-release formulation.

12. The method of claim 1, wherein the disease of the intervertebral disc is degenerative disc disease.

13. The method of claim 1, wherein the disease is characterized by an increased amount of at least one heteropolysaccharide.

14. The method of claim 13 wherein the at least one heteropolysaccharide is a glycosaminoglycan.

15. The method of claim 14, wherein the glycosaminoglycan is a sulfated-glycosaminoglycan.

16. The method of claim 1, wherein the LMP protein is selected from the group consisting of an LMP-1 protein, an LMP-2 protein, an LMP-3 protein, an LMP-1s protein and any combination thereof.

17. The method of claim 1, wherein the composition further comprises transportation means.

18. The method of claim 17, wherein the transportation means comprises an amino acid sequence encoding a TAT or a PTD domain.

19. The method of claim 1, further comprising the step of harvesting the cell from a patient having the disease of the intervertebral disc or from an immunologically compatible donor.

20. The method of claim 19, wherein the cell is a stem cell.

21. The method of claim 20, wherein the stem cell is an adult stem cell.

22. The method of claim 21, wherein the adult stem cell is derived from blood, bone marrow, adipose tissue, muscle tissue, brain tissue, or any combination thereof.

23. The method of claim 20, wherein the stem cell is cultured under conditions promoting differentiation of the stem cell into a cell of an intervertebral disc.

24. The method of claim 19, wherein the cell is an intervertebral disc cell.

25. The method of claim 19, wherein the step of increasing the amount of the at least one proteoglycan, at least one collagen, or the at least one heteropolysaccharide in the cell is performed in vitro.

26. The method of claim 25, further comprising the step of administering the cell into the intervertebral disc having the disease.

27. The method of claim 1, wherein the step of increasing the amount of the at least one proteoglycan, at least one collagen, or the at least one heteropolysaccharide in the cell is performed in vivo.

28. The method of claim 1, wherein the step of increasing the amount of the at least one proteoglycan, at least one collagen, or the at least one heteropolysaccharide in the cell comprises administering the composition to the intervertebral disc having the disease.

29. The method of claim 28, wherein the composition is included within an implant.

30. The method of claim 1, wherein the composition further comprises at least one additive.

31. The method of claim 1, wherein the composition is a sustained-release composition.


SEQUENCE LISTING (19 sequences)

SEQ ID NO: 1 - protein, 457 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 2 - protein, 423 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKVQTPDKQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 3 - protein, 153 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGCRPLTNSRSDRWSQMPASSG

SEQ ID NO: 4 - protein, 223 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPP

SEQ ID NO: 5 - protein, 9 aa, Artificial
  Description of Artificial Sequence Synthetic Peptide
RKKRRQRRR

SEQ ID NO: 6 - protein, 16 aa, Artificial
  Description of Artificial Sequence Synthetic Peptide
RQIKIWFQNRRMKWKK

SEQ ID NO: 7 - protein, 14 aa, Artificial
  Description of Artificial Sequence Synthetic Peptide
GRKKRRQRRRPPQC

SEQ ID NO: 8 - protein, 26 aa, Artificial
  Description of Artificial Sequence Synthetic Peptide
GWTLNSAGYLLKINLKALAALAKKIL

SEQ ID NO: 9 - protein, 9 aa, Artificial
  Description of Artificial Sequence Synthetic Peptide
RRRRRRRRR

SEQ ID NO: 10 - protein, 12 aa, Artificial
  Description of Artificial Sequence Synthetic Peptide
PIRRRKKLRRLK

SEQ ID NO: 11 - protein, 12 aa, Artificial
  Description of Artificial Sequence Synthetic Peptide
RRQRRTSKLMKR

SEQ ID NO: 12 - protein, 12 aa, Artificial
  Description of Artificial Sequence Synthetic Peptide
SRRKRQRSNMRI

SEQ ID NO: 13 - protein, 12 aa, Artificial
  Description of Artificial Sequence Synthetic Peptide
SFHQFARATLAS

SEQ ID NO: 14 - protein, 12 aa, Artificial
  Description of Artificial Sequence Synthetic Peptide
DPATNPGPHFPR

SEQ ID NO: 15 - protein, 12 aa, Artificial
  Description of Artificial Sequence Synthetic Peptide
TLPSPLALLTVH

SEQ ID NO: 16 - DNA, 1644 bp, Homo sapiens
atggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctgaattccaaaaaatccaaaaaaaaa

SEQ ID NO: 17 - DNA, 1567 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 18 - DNA, 1686 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatgggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 19 - DNA, 1667 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgagctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctgvv